(19) United States
(12) Patent Application Publication
(10) Pub. No.: US 2002/0076354 A1
(43) Pub. Date: Jun. 20, 2002

(54) Apparatus and methods for separating components of particulate suspension

(76) Inventor: David Samuel Cohen, Alpharetta, GA (US)

Correspondence Address: HALE AND DORR, LLP, 60 STATE STREET, BOSTON, MA, 02109

(21) Appl. No.: 09/997,895

(22) Filed: Nov. 30, 2001

Related U.S. Application Data
(60) Provisional application No. 60/250,588, filed on Dec. 1, 2000.

(51) Int. Cl. G01N 21/07; C12Q 1/68; G01N 33/53; G01N 33/542; C12M 1/34

(52) U.S. Cl. 422/72; 435/6; 435/287.2; 435/7.9; 422/82.05; 422/101

(57) ABSTRACT

An optical bio-disc used in separating components of particulate suspension has separation, fluid metering, and fluid assay chambers. The separation chamber contains a particulate suspension having a fluid component and a particulate matter component. The fluid metering chamber communicates with the separation chamber by a first conduit that has an entry point at the separation chamber. The entry point is accessible to the fluid component when the bio-disc is rotated causing separation of the fluid component and the particulate matter component in the separation chamber. The fluid assay chamber communicates with the fluid metering chamber by a second conduit.

### CROSS-REFERENCE TO RELATED APPLICATIONS

This application claims the benefit of U.S. Provisional Application No. 60/250,588, entitled MICROFLUIDIC CIRCUIT FOR SEPARATING AND METERING FLUID COMPONENTS FROM A PARTICULATE SUSPENSION AND OPTICAL BIO-DISC AND DRIVE ASSEMBLY RELATING THERETO, filed on Dec. 1, 2000, which is incorporated by reference.

### BACKGROUND

This invention relates to separating components of particulate suspension.

A particulate suspension has a fluid component mixed with a particulate matter component. For example, blood is a particulate suspension having red and white blood cells suspended in plasma. U.S. Pat. No. 6,063,589 to Kellogg et al. (“Kellogg”) describes a rotating bio-disc platform having a microfluidic array for separating a fluid component from a particulate suspension. Kellogg FIGS. 7-9 illustrate a microsystems platform for separating vertebrate blood cells and components. The components of Kellogg's array include an entry port constructed on the platform to accommodate a volume of about 5 to about 50 microliters. The entry port is fluidly connected to an entry capillary having a size sufficient to contain a total volume of from about 1 to about 15 microliters. The entry capillary is further fluidly connected to a separation column having a size sufficient to contain a total volume of 10 to about 20 microliters. The separation column is also fluidly connected with a passage to an overflow chamber. A small capillary exit is also fluidly connected with the separation chamber and is arranged to traverse a direction radially more proximal to the axis of rotation than the insertion point with the separation column. The small capillary terminates in a capillary junction that is fluidly connected with the capillary, extending in a radial direction to a decant chamber. A sacrificial valve is positioned in a small capillary at the juncture with the capillary junction. The capillary is arranged in a radially outward direction between the capillary junction and the decant chamber. The passage is positioned on the separation column to be significantly more proximal to the axis of rotation than the insertion point of the small capillary.

Kellogg's platform is described in use for separating plasma from whole blood. An imprecise volume (ranging from 1-150 microliters) of blood is applied to the entry port. Blood enters the entry capillary by capillary action, and stops at the capillary junction between the entry capillary and the separation chamber. At a first rotational speed, blood flows from the entry capillary into the separation chamber. Blood continues to fill the separation column until blood reaches the position of the passage, whereupon excess blood flows through the passage into the overflow chamber. After a sufficient time of rotation at the first non-zero rotational speed, the excess blood has been transferred into the overflow chamber and the separation column is filled with blood to the position of the passage. Rotation at a second rotational speed that is greater than the first rotational speed separates blood components into red blood cell, white blood cell (“buffy coat”), and plasma fractions. The dimensions of the small capillary permit fluid flow of the plasma fraction through the capillary that is stopped at the capillary junction. Fluid flow of plasma into the decant chamber results from fluid flow overcoming the capillary barrier by rotation at a third rotational speed that is greater than the second rotational speed.

As illustrated in Kellogg FIGS. 12A-12J, Kellogg also describes an alternative embodiment of the fluid separation platform, particularly a blood separation microfluidics array. The array has an entry port that is capable of accommodating a volume of about 5 to about 50 microliters and is fluidly connected with a first array of metering capillaries and a second array of metering capillaries. The first metering capillary array is fluidly connected with a ballast chamber, wherein the first metering capillary array forms a capillary junction between the array and the ballast chamber. The second capillary array is fluidly connected with a capillary junction. The entry port is also constructed to be fluidly connected with an overflow capillary that is fluidly connected with an overflow chamber.

The ballast chamber acts as a capillary barrier that prevents fluid flow from the first metering capillary array at a first, non-zero rotational speed sufficient to permit fluid flow comprising excess blood overflow from the entry port through the overflow capillary and into the overflow chamber. The capillary junction is a capillary barrier that prevents fluid flow from the second metering capillary array at the first, non-zero rotational speed.

The ballast chamber is fluidly connected to a capillary that is connected to a capillary junction. Alternatively, the capillary is fluidly connected with a sacrificial valve. The capillary junction or sacrificial valve is further fluidly connected with a channel which is fluidly connected with a separation chamber at a point most distal from the axis of rotation.

The second metering capillary array is fluidly connected with a capillary junction that is overcome at a second, higher rotational speed. The capillary junction is further fluidly connected to a channel which is further fluidly connected to the separation chamber. A channel extends from capillary junction to the separation chamber which is fluidly connected with a decant channel at a point close to the chamber's most axis-proximal extent. The decant channel is fluidly connected with a decant chamber.

In a use of the embodiment, an imprecise volume (ranging from 1-150 microliters) of blood is applied to the entry port. Blood enters the each of the metering capillary arrays and stops at the capillary junction between the first metering capillary array and the ballast chamber and between the second metering capillary and the corresponding capillary junction. Blood also enters and fills the overflow capillary, stopping at the capillary junction with the overflow chamber. At a first rotational speed, blood flows from the entry port through overflow capillary and into the overflow chamber. At a second rotational speed greater than the first rotational speed, the capillary junction between the first metering capillary array and the ballast chamber is overcome, and blood from the first metering capillary array fills the ballast chamber. Similarly, at the second rotational speed, the corresponding capillary junction is overcome, and blood from the second metering capillary array enters the separation chamber. The volume of blood in the second metering capillary array is insufficient to fill the separation chamber to the level of insertion of the decant channel.

By rotation at a third rotational speed that is greater than the second rotational speed, blood components in the separation chamber are separated into red blood cell, white blood cell, and plasma fractions. Separation of blood components is not achieved in the ballast chamber due to its position on the platform, and the corresponding capillary junction or sacrificial valve is not overcome at the third rotational speed. The separated plasma does not extend to the decant capillary.

Release of the sacrificial valve, or rotation at a fourth rotational speed that is greater than the third rotational speed, results in flow of blood from the ballast chamber through the corresponding channel into the separation chamber at the “bottom” or most rotation-axis-distal extent of the separation chamber. This results in the filling of the separation chamber to a position equal to the insertion point of the decant channel. Plasma flows through the decant channel into the decant chamber in an amount equal to the amount of blood contained in the ballast chamber. The decant channel has dimensions that retard passage of unfractionated blood, or plasma contaminated with greater than 0.1-1% of blood cells found in whole blood.

Thus, these embodiments show systems in which centrifugal force is used in varying degrees in a fluidic circuit to cause blood cells of a blood specimen to concentrate in a particular portion of a first component of the circuit, and to cause blood serum of the specimen to be decanted directly from the first component to a second component that serves as the destination for the serum.

### SUMMARY OF THE INVENTION

The embodiments of the present invention include microfluidic circuits, optical bio-discs, systems and methods for separating particulate components from a fluid component in a particulate suspension, and systems and methods for analyzing fluid and particulate components of a particulate suspension.

A microfluidic circuit used for separating components of particulate suspension has separation, fluid metering, and fluid assay chambers, preferably formed in the substrate of a disc, preferably the size of a compact disc (CD) and designed to be read in an optical disc reader. The separation chamber contains a particulate suspension having a fluid component and a particulate matter component. The fluid metering chamber communicates with the separation chamber by a first conduit that has an entry point at the separation chamber. The entry point is accessible to the fluid component when the bio-disc is rotated causing separation of the fluid component and the particulate matter component in the separation chamber. The fluid assay chamber communicates with the fluid metering chamber by a second conduit.

The assay chamber is preferably located at a target zone, or viewing window, where a light source in an optical reader can detect some aspect of the fluid, through one of a number of detection methods.

The embodiments also include a metering chamber such as a loop formed in a substrate of a rotated bio-disc for receiving a liquid during a first rotation step, and for delivering the liquid to another chamber (e.g., an assay zone) in the substrate in a second rotating step. The metering chamber may be U-shaped with a bight portion at the outermost radial point of the chamber, and may be fully or nearly symmetric about a radius from the axis of rotation of the disc.

The embodiments also include a separation chamber that is elongated and is at an acute angle relative to a vector of rotation-induced centrifugal force through the center of the separation chamber. The example embodiment shows this angle as approximately 30 degrees, but the angle could be in a range of 0 to 45 degrees.

Implementations of the systems and methods of the invention may provide one or more of the following advantages. A predictable and controllable amount of a fluid component can be automatically extracted from a particulate suspension and can be automatically directed to an assay zone in preparation for analysis. An assay can be performed on the fluid component of a particulate suspension quickly and inexpensively under controlled conditions. Results of the assay can be determined automatically and data representing the results can be gathered, stored, and distributed electronically and automatically. Inexpensive equipment using existing technology can be enhanced to provide rapid and automatic separation of fluid and particulate matter components of a specimen. An analysis can be performed that produces results that are relative to a known volume of a fluid component, e.g., a concentration of a hormone in blood serum.

Other advantages and features will become apparent from the following description, including the drawings, and from the claims.

### BRIEF DESCRIPTION OF THE DRAWINGS

FIG. 1A is an exploded view of a reflective bio-disc that may be employed in connection with one or more aspects of the present invention.

FIG. 1B is a top view of the reflective bio-disc as illustrated in FIG. 1A.

FIG. 1C is a perspective view of the reflective bio-disc as illustrated in FIGS. 1A-1B.

FIG. 2A is an exploded view of a transmissive bio-disc that may be utilized in conjunction with one or more aspects of the present invention.

FIG. 2B is a top view of the transmissive bio-disc as illustrated in FIG. 2A.

FIG. 2C is a perspective view of the transmissive bio-disc as illustrated in FIGS. 2A-2B.

FIG. 3 is a block diagram of an optical reading system that may be used in connection with one or more aspects of the present invention.

FIG. 4 is a perspective view of an embodiment of a bio-disc and an optical reading system implemented in accordance with one or more apsects of the present invention.

FIG. 5 is a plan view of one embodiment of a bio-disc having a microfluidic circuit in accordance with one or more aspects of the present invention.

FIG. 6 is a plan view of another embodiment of a bio-disc having a microfluidic circuit in accordance with one or more aspects of the present invention.

FIG. 7 is a plan view of yet another an embodiment of a bio-disc having a microfluidic circuit in accordance with one or more aspects of the present invention.

### DETAILED DESCRIPTION

An optical bio-disc for use with embodiments of the present invention may have any suitable shape, diameter, or thickness, but preferably is implemented on a round disc with a diameter and a thickness similar to those of a compact disc (CD), a recordable CD (CD-R), CD-RW, a digital versatile disc (DVD), DVDR, DVD-RW, or other standard optical disc format. The disc may include encoded information, preferably in a known format, for performing, controlling, and post-processing a test or assay, such as information for controlling the rotation rate of the disc, timing for rotation, stopping and starting, delay periods, multiple rotation steps, locations of samples, position of the light source, and power of the light source. Such encoded information is referred to generally as operational information.

The disc may be reflective, transmissive, or some combination of reflective and transmissive. In the case of a reflective disc, an incident light beam is focused onto or directed to a reflective surface of the disc, reflected, and returned through optical elements to a detector on the same side of the disc as the light source. In a transmissive disc, light passes through the disc (or portions thereof) to a detector on the other side of the disc from the light source. In a transmissive portion of a disc, some light may also be reflected and detected as reflected light. The light may include any type of electromagnetic radiation, such as visible light, infrared light, or ultraviolet light.

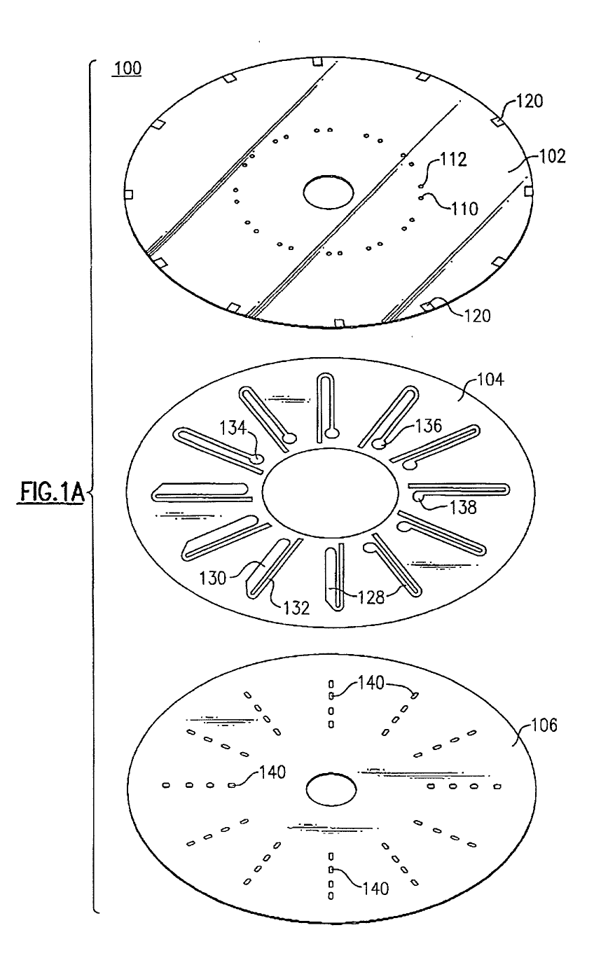

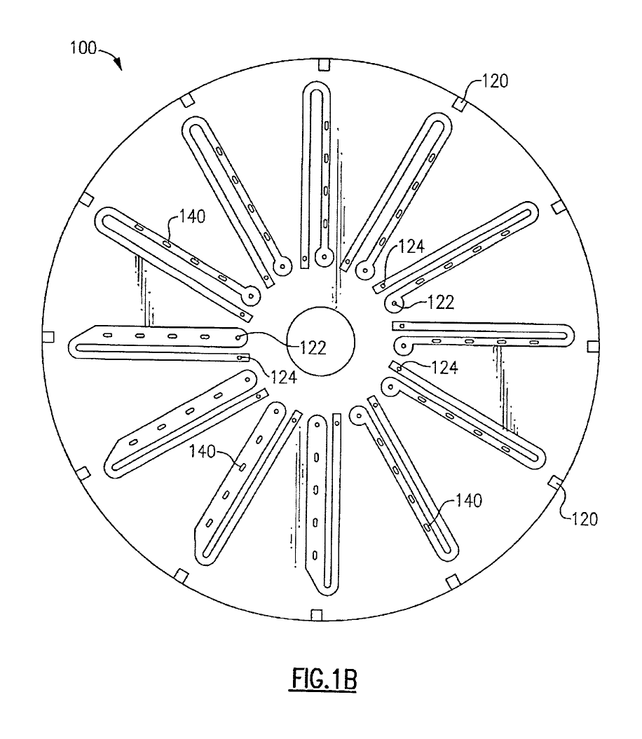

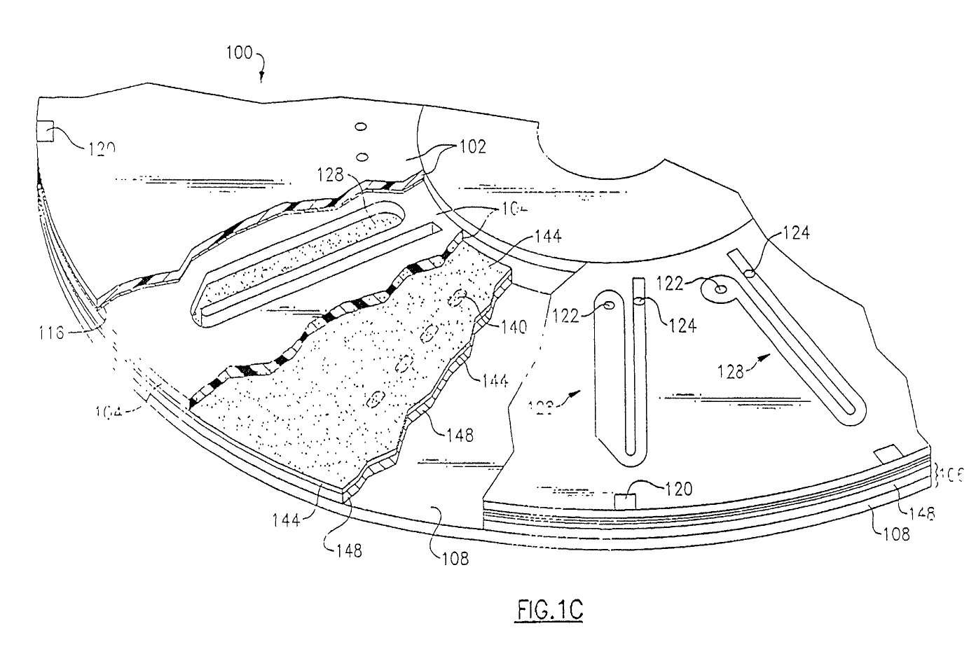

Referring to FIGS. 1A, 1B, and 1C, a reflective disc 100 is shown with a cap 102, a channel layer 104, and a substrate 106. Cap 102 has inlet ports 110 for receiving samples and vent ports 112. Cap 102 may be formed primarily from a material such as polycarbonate, and may be coated with a reflective layer 116 on the bottom thereof. Reflective layer 116 is preferably made from a metal, such as aluminum or gold.

Channel layer 104 defines fluidic circuits 128 by having desired shapes from channel layer 104. (As described in more detail below, one or more of circuits 128 may be replaced by, e.g., circuit 412 of FIG. 5.) Each fluidic circuit 128 preferably has a flow channel 130 and a return vent channel 132, and some have a mixing chamber (e.g., chamber 134). A mixing chamber 136 can be symmetrically formed relative to the flow channel 130, while an off-set mixing chamber 138 is formed to one side of the flow channel 130. Fluidic circuits 128 can include other channels and chambers, such as preparatory regions or a waste region, as shown, for example, in U.S. Pat. No. 6,030,581, which is incorporated herein by reference. Channel layer 104 can include adhesives for bonding substrate to cap.

Substrate 106 has a non-conductive (e.g., polycarbonate) layer 108, and has target zones 140 formed as openings in a reflective layer 148 deposited on the top of layer 108. Target zones 140 may be formed by removing portions of reflective layer 148 in any desired shape, or by masking target zone areas before applying reflective layer 148. Reflective layer 148 is preferably formed from a metal, such as aluminum or gold, and can be configured with the rest of the substrate to encode operational information that is read with incident light, such as through a wobble groove or through an arrangement of pits. Light incident from under substrate 106 thus is reflected by layer 148, except at target zones 140, where it is reflected by layer 116. Target zones may have imaged features without capture, while a capture zone generally refers to a location where an antibody or other anti-ligand is located.

Referring particularly to FIG. 1C, optical disc 100 is cut away to illustrate a partial cross-sectional view. An active capture layer 144 is formed over reflective layer 148. Capture layer 144 may generally be formed from nitrocellulose, polystyrene, polycarbonate, gold, activated glass, modified glass, or a modified polystyrene, for example, polystyrene-co-maleic anhydride. Channel layer 104 is over capture layer 144. Polystyrene is generally preferred for a WBC capture zone.

Trigger marks 120 are preferably included on the surface of a reflective layer 148, and may include a clear window in all three layers of the disc, an opaque area, or a reflective or semi-reflective area encoded with information. These are discussed below.

In operation, samples are introduced through inlet ports 110 of cap 102. When rotated, the sample moves outwardly from inlet port 110 along capture layer 144. Through one of a number of biological or chemical reactions or processes, detectable features may be present in the target zones. These features are referred to as investigational features. Examples of such processes are shown in the incorporated U.S. Pat. No. 6,030,581.

The investigational features captured by the capture layer may be designed to be located in the focal plane coplanar with reflective layer 148, where an incident beam is typically focused in conventional readers; alternatively, the investigational features may be captured in a plane spaced from the focal plane. The former configuration is referred to as a “proximal” type disc, and the latter a “distal” type disc.

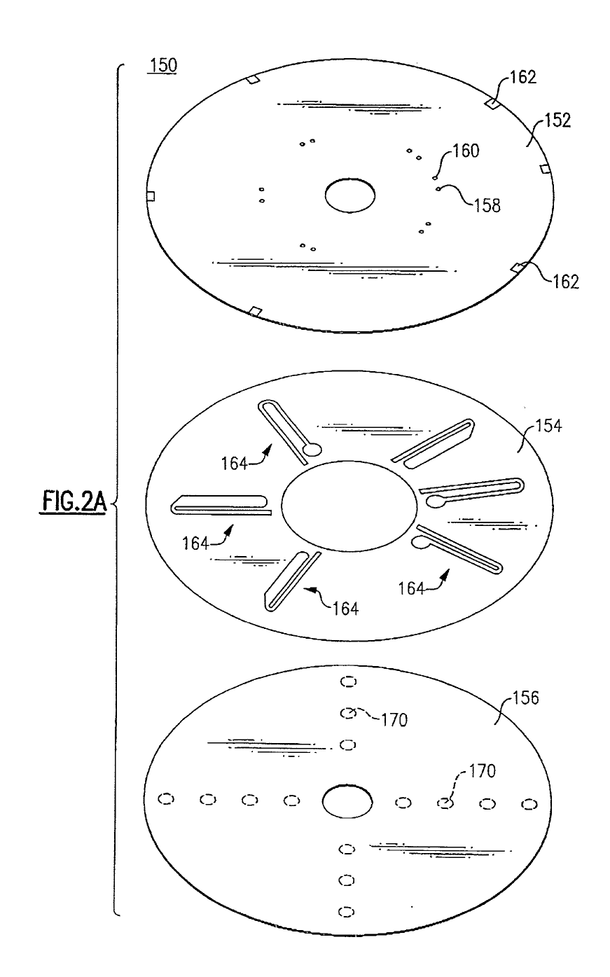

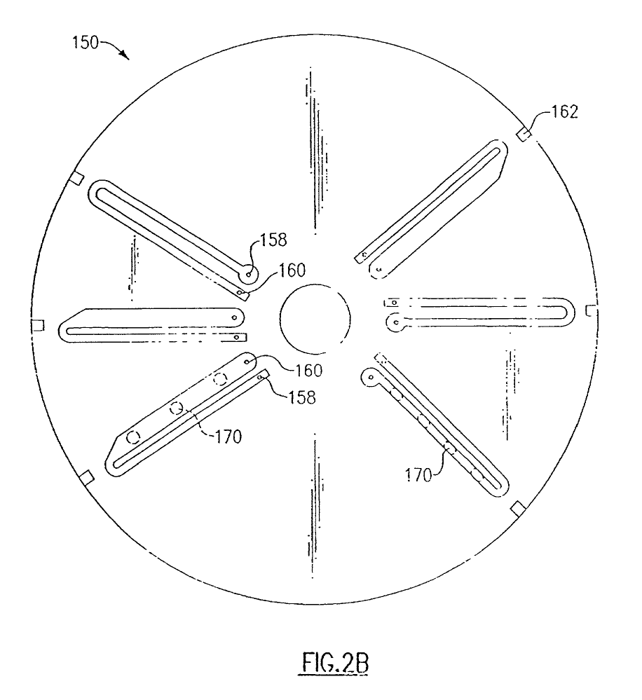

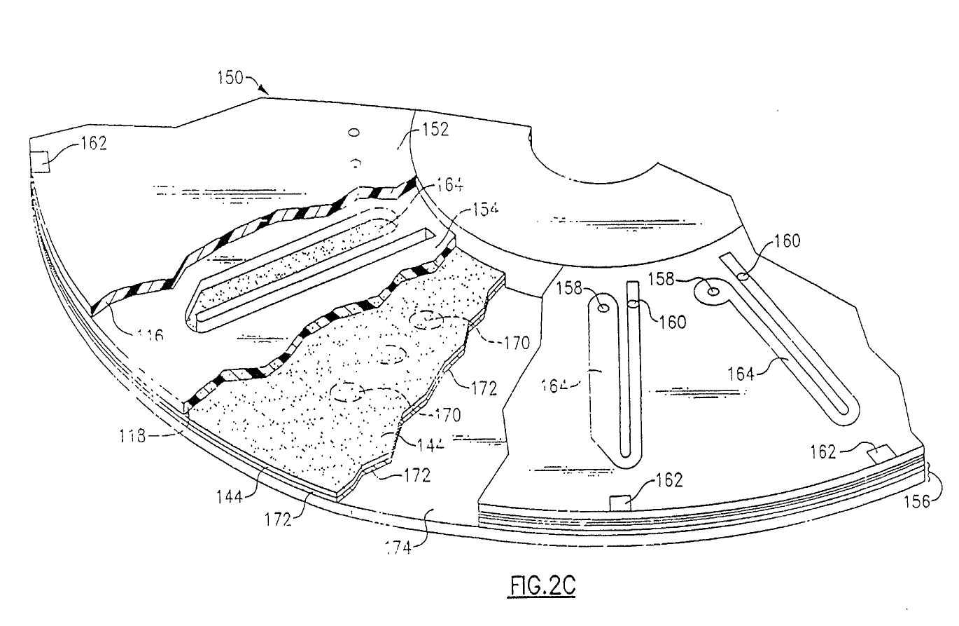

Referring to FIGS. 2A, 2B, and 2C, a transmissive or semi-reflective optical disc 150 has a cap 152, a channel layer 154, and a substrate 156. Cap 152 includes inlet ports 158 and vent ports 160 and is preferably formed mainly from polycarbonate. Trigger marks 162 similar to those for disc 100 may be included. Channel layer 154 has fluidic circuits 164, which can have structure and use similar to those described in conjunction with FIGS. 1A, 1B, and 1C. (As described in more detail below, one or more of circuits 164 may be replaced by, e.g., circuit 412 of FIG. 5.) Substrate 156 may include target zones 170, and preferably includes polycarbonate layer 174. Substrate 156 may, but need not, have a thin semireflective layer 172 deposited on top of layer 174. Semi-reflective layer 172 is preferably significantly thinner than reflective layer 148 on substrate 106 of reflective disc 100 (FIGS. 1A-1C). Semi-reflective layer 172 is preferably formed form a metal, such as aluminum or gold, but is sufficiently thin to allow a portion of an incident light beam to penetrate and pass through layer 172, while some of the incident light is reflected back. A gold film layer, for example, is 95% reflective at a thickness greater than about 700 Å, while the transmission of light through the gold film is about 50% transmissive at approximately 100 Å.

FIG. 2C is a cut-away perspective view of disc 150. The semi-reflective nature of layer 172 makes its entire surface available for target zones, including virtual zones defined by trigger marks or specially encoded data patterns on the disc. Target zones 170 may also be formed by marking the designated area in the indicated shape or alternatively in any desired shape. Markings to indicate target zone 170 may be made on semi-reflective layer 172 or on a bottom portion of substrate 156 (under the disc). Target zones 170 may be created by silk screening ink onto semi-reflective layer 172.

An active capture layer 180 is applied over semi-reflective layer 172. Capture layer 180 may be formed from the same materials as described above in conjunction with layer 144 (FIG. 1C) and serves substantially the same purpose when a sample is provided through an opening in disc 150 and the disc is rotated. In transmissive disc 150, there is no reflective layer comparable to reflective layer 116 in reflective disc 100 (FIG. 1C).

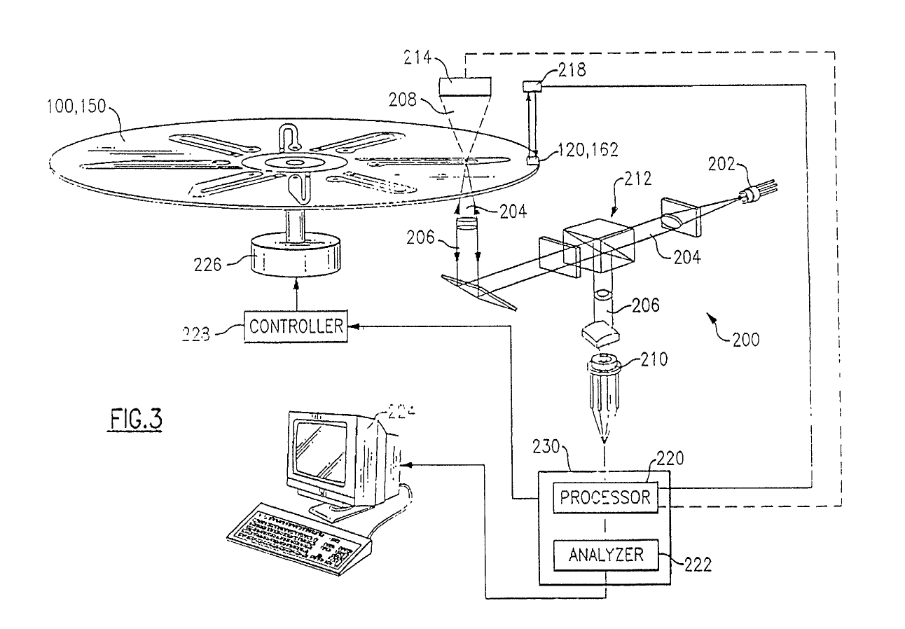

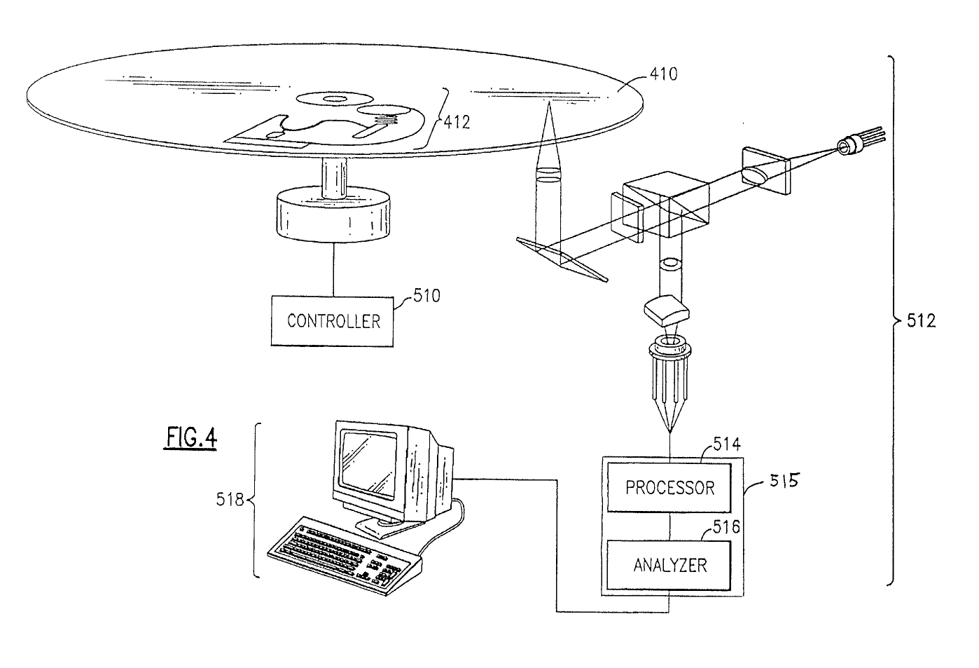

FIG. 3 shows an optical disc reader system 200. This system may be a conventional reader for CD, CD-R, DVD, or other known comparable format, a modified version of such a drive, or a completely distinct dedicated device. The basic components are a motor for rotating the disc, a light system for providing light, and a detection system for detecting light.

A light source 202 provides light to optical components 212 to produce an incident light beam 204, a return beam 206, and a transmitted beam 208. In the case of reflective disc 100, return beam 206 is reflected from either reflective surface 148 or 116. Return beam 206 is provided back to optical components 212, and then to a bottom detector 210. For transmissive disc 150, a transmitted beam 208 is detected by a top detector 214. Optical components 212 can include a lens, a beam splitter, and a quarter wave plate that changes the polarization of the light beam so that the beam splitter directs a reflected beam through the lens to focus the reflected beam onto the detector. An astigmatic element, such as a cylindrical lens, may be provided between the beam splitter and detector to introduce astigmatism in the reflected light beam.

Data from detector 210 and/or detector 214 is provided to a computer 230 including a processor 220 and an analyzer 222. An image or output results can then be provided to a monitor 224. Computer 230 can represent a desktop computer, programmable logic, or some other processing device, and also can include a connection (such as over the Internet) to other processing and/or storage devices. A drive motor 226 and a controller 228 are provided for controlling the rotation and direction of disc 100 or 150. Controller 228 and the computer with processor 220 can be in communication or can be the same computer. Methods and systems for reading such a disc are also shown in Gordon, U.S. Pat. No. 5,892,577, which is incorporated herein by reference.

A hardware trigger sensor 218 may be used with either a reflective or transmissive disc. Triggering sensor 218 provides a signal to computer 230 (or to some other electronics) to allow for the collection of data by processor 220 only when incident beam 204 is on a target zone. Alternatively, software read from a disc can be used to control data collection by processor 220 independent of any physical marks on the disc.

The substrate layer may be impressed with a spiral track that starts at an innermost readable portion of the disc and then spirals out to an outermost readable portion of the disc. In a non-recordable CD, this track is made up of a series of embossed pits with varying length, each typically having a depth of approximately one-quarter the wavelength of the light that is used to read the disc. The varying lengths and spacing between the pits encode the operational data. The spiral groove of a recordable CD-like disc has a detectable dye rather than pits. This is where the operational information, such as the rotation rate, is recorded. Depending on the test, assay, or investigational protocol, the rotation rate may be variable with intervening or consecutive periods of acceleration, constant speed, and deceleration. These periods may be closely controlled both as to speed and time of rotation to provide, for example, mixing, agitation, or separation of fluids and suspensions with agents, reagents, antibodies, or other materials.

Numerous designs and configurations of an optical pickup and associated electronics may be used in the context of the embodiments of the present invention. Further details and alternative designs for compact discs and readers are described in Compact Disc Technology, by Nakajima and Ogawa, IOS Press, Inc. (1992); The Compact Disc Handbook, Digital Audio and Compact Disc Technology, by Baert et al. (eds.), Books Britain (1995); and CD-Rom Professional's CD-Recordable Handbook: The Complete Guide to Practical Desktop CD, Starrett et al. (eds.), ISBN:0910965188 (1996); all of which are incorporated herein in their entirety by reference.

The disc drive assembly is thus employed to rotate the disc, read and process any encoded operational information (e.g., analysis instructions) stored on the disc, analyze the liquid, chemical, biological, or biochemical investigational features in an assay region of the disc, to write information (e.g., analysis identifiers or results) to the disc either before, during, and/or after the material in the assay zone is analyzed by the read beam of the drive or deliver the information via various possible interfaces, such as Ethernet to a user, database, or anywhere the information could be utilized.

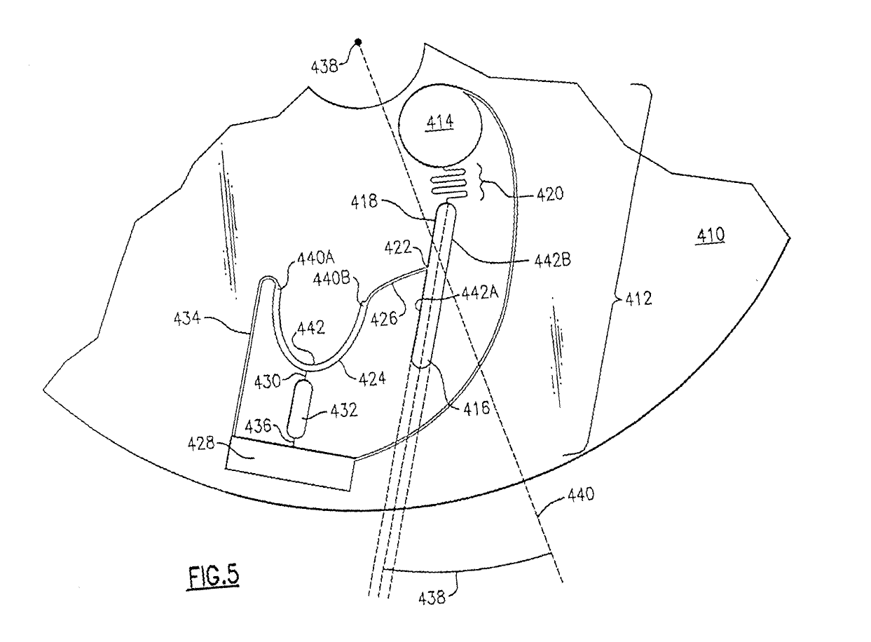

An optical bio-disc such as the disc described above may have one or more microfluidic circuits that perform any of various functions. For example, a microfluidic circuit may be used for separating or otherwise manipulating components of a particulate suspension. FIG. 4 illustrates an example of an optical bio-disc 410 of a type having a microfluidic circuit 412 for separating and metering a fluid component from a particulate suspension. A controller 510 controls the rotation of the optical bio-disc. An optical disc reader system 512 including a computer 515 having a processor 514 and an analyzer 516, and a monitor with a display 518 is provided to process and analyze optical signals from the optical bio-disc and present results of the processing and analysis. For example, the system may read encoded information from the optical bio-disc and may analyze the fluid component separated by the circuit. Computer 515 or monitor 518 or both computer 515 and monitor 518 can be in communication with controller 510 or could be the same computer, e.g., such that the read data either governs operation of the drive, or is used to cause the disk to do additional tasks.

In particular, as described below, when a particulate suspension including a particulate matter component and a fluid (e.g., liquid) component is deposited into an antechamber of the circuit, rotating the bio-disc in a predetermined manner delivers a metered amount of the fluid component to an assay zone of the circuit.

In a case in which the microfluidic circuit is initially filled with whole or diluted blood, for example, the circuit can separate at least some of the blood's serum component from the blood's cellular components (particulate matter including white blood cells and red blood cells) and deliver a metered amount of the serum component to an assay zone.

The circuit is mounted on a rotatable platform such that rotation produces centrifugal force to move the blood throughout the circuit. As noted above, the rotatable platform may include an optical bio-disc such as the bio-disc of FIGS. 1A-1C or the bio-disc of FIGS. 2A-2C, and the optical bio-disc may be reflective or transmissive and may include one or more aspects of the present invention as described herein in connection with microfluidic circuits shown in FIGS. 4-7. The optical bio-disc may be implemented on an optical disc including a format such as standard or modified CD, CD-R, or DVD. The bio-disc may include encoded information, which may be used, for example, for controlling the rotation rate of the disc. The rotation rate is variable and is controlled as to speed or duration of rotation or both. A bio-disc drive assembly is employed to rotate the disc, read and process any encoded information stored on the disc, and analyze the specimen in the assay zone. The bio-disc drive assembly may also be utilized to write information to the bio-disc either before or after the material in the assay zone is analyzed by the read beam of the drive.

FIG. 5 shows that microfluidic circuit 412 includes several substructures, each of which has a role in the circuit's processing. An antechamber 414 is of sufficient size to accommodate the entire sample volume (e.g., approximately 10 microliters). In the case of blood, blood is initially injected into the antechamber and the platform is spun at a speed s1 for a time t1. Antechamber 414 may be pre-loaded with a material that includes a freeze-dried anticoagulant such as ethylene diamine tetra-acetic acid (EDTA) or sodium citrate to help prevent coagulation of the sample during processing or analysis in the circuit. Upon entry of the sample into the antechamber, the freeze-dried anticoagulant dissolves into the sample. In at least some cases, the volume of anticoagulant that is provided and that is dissolved amounts to much less than 1% of the volume of the sample that is directed further downstream in the circuit.

As a result of the spinning, the blood moves from the antechamber to a separation chamber 418 (e.g., a separation tube) and the blood's cellular components are urged toward the non-entrance end 416 of the chamber. The antechamber and the separation chamber are in fluid communication by use of a first conduit 420. Centrifugal force resulting from speed s1 is insufficient to overcome capillary forces at junction 422 between the separation chamber and a metering chamber 424 (e.g., a metering loop tube). (Chamber 424 may include a chamber of nearly any shape having an input and an output, as long as a controlled amount of fluid is retained in the chamber after termination of a stage of fluid flow through the chamber. For example, a set of multiple chambers may serve as the metering chamber.)

In at least some embodiments, the location of junction 422 on the separation chamber is a function of the expected composition of the particulate suspension, and is selected for access to the fluid component of the suspension. For example, in the case of blood which is expected to be approximately 50% particulate (e.g., blood cells and platelets, or only blood cells) by volume, junction 422 is disposed at a point corresponding to slightly more than 50% of the volume of the separation chamber, as measured from outward end of the chamber (i.e., the end that is furthest from the center of rotation). Thus, junction 422 is located to provide access to all or nearly all of the remainder (e.g., serum, or serum and platelets) without being blocked by the particulate that collects at the outward end.

As shown in FIG. 5, the first conduit 420 may have a zigzag pattern shape in which different legs of the conduit in the pattern carry particulate suspension in substantially opposite directions on the route between the antechamber and the separation chamber. Such a pattern shape improves mixing (e.g., of anticoagulant with blood) by causing increased turbulence during downstream flow.

The separation chamber and the metering chamber are in fluid communication by use of a second conduit 426. At the end of time t1 the speed is increased to a speed s2 and held for a time t2. The resulting increased centrifugal force is sufficient to overcome the capillary forces at junction 422, and moves serum out of the separation chamber and into the metering chamber and overflow conduit (e.g., loop) 434. The centrifugal forcing resulting from speed s2 is insufficient to overcome the capillary forces at junction 430 between the metering chamber and assay zone 432. Serum volume that is beyond the capacity of the metering chamber is directed through the overflow conduit into waste chamber 428. At the end of time t2 the speed is increased to speed s3 and held for time t3. The resulting centrifugal force moves the serum out of the metering chamber and into the assay zone, but is insufficient to overcome the capillary forces at junction 436 between the assay zone and the waste chamber. In addition, this rotation is done such that the serum in the overflow conduit is not drawn back into the metering chamber and then the assay zone. This means that the amount of fluid provided to the assay zone will be the amount of fluid held in the metering chamber (assuming there was enough in the first place), even if the quantity input is much greater than the volume of the metering chamber.

The optical bio-disc may have encoded information including instructions for controlling such rotation, or other handling, of the bio-disc or the microfluidic circuit. Thus, the data on the disc can be read to cause the disc to be rotated at a particular speed for a particular time, wait, and then rotate at a next particular speed for a next particular time.

As shown in FIG. 5, the fluid metering chamber may have a U shape or an elongated shape and the axis of rotation 438 may be closer to the ends 440A, 440B of the fluid metering chamber than the axis of rotation is to the middle 442 of the fluid metering chamber. As shown, the U-shape has a bight portion at its radially outermost point, and is symmetric about a radial line perpendicular to the axis of rotation. (As noted above, in at least some cases, any chamber or set of chambers having particular characteristics noted above may serve as the metering chamber. For example, in at least some cases, it is not necessary for the metering chamber to have a U-shape or be symmetric about any particular line.)

As described above, the circuit may be used to process a particulate suspension including blood having red and white blood cells and platelets suspended in serum. The circuit may also be used for processing other biological particulate suspensions such as urine, environmental water, amniotic fluid, cerebrospinal fluid, synovial fluid, pleural fluid, pericardial fluid, peritoneal fluid, saliva, and semen, and for processing chemical solutions and suspensions. In the case of a urine sample, the particulate matter component may include epithelial cells, casts, or bacteria, and the fluid component may include clarified urine. In the case of an environmental water sample, the particulate matter component may include dirt, biological matter, particulate contaminants, or bacteria, and the fluid component may include clarified water. In the case of amniotic fluid, the particulate matter component may include sloughed cells, cell debris, cells, vernix, or bacteria, and the liquid component may include clarified amniotic fluid. In the case of cerebrospinal fluid, the particulate matter component may include cell debris, cells, clots, or bacteria, and the fluid component may include clarified cerebrospinal fluid. In the case of synovial fluid, the particulate matter component may include cell debris, cells, clots, or bacteria, and the liquid component may include clarified synovial fluid. In the case of pleural fluid, the particulate matter component may include cell debris, cells, lipid, or bacteria, and the liquid component may include clarified pleural fluid. In the case of pericardial fluid, the particulate matter component may include cell debris, cells, lipid, or bacteria, and the liquid component may include clarified pericardial fluid. In the case of peritoneal fluid, the particulate matter component may include cell debris, cells, lipid, and bacteria, and the fluid component may include clarified peritoneal fluid.

In a specific implementation shown in FIG. 5, the separation chamber is oriented such that its centerline is at an angle 438 (e.g., approximately 30 degrees) to a vector 440 of rotation-induced centrifugal force through the center of the separation chamber. In particular, opposite boundaries 442A, 442B (shown substantially in parallel in FIG. 5) of the separation chamber may be 0-45 degrees (e.g., approximately 30 degrees as shown) in the same direction from the vector. (At an angle greater than 45 degrees, the separation chamber may cause conduit 436 to have an orientation that leads fluid toward the center of rotation, which could hamper the desired movement of the fluid, instead of downstream toward the metering chamber.) Such angling of the separation chamber helps to decrease the possibility of cellular component contamination in the serum that is moved to the metering chamber.

In a specific embodiment, platform-speed-controlled valves are employed in the microfluidic circuit to connect the separation chamber to the metering chamber and to connect the metering chamber to the assay zone so that appropriate fluid communication is provided therebetween. The platform-speed controlled valves operate by supplying differing capillary pressures across respective changes in the cross-sectional dimensions of adjoining channels or substructures.

Once the fluid reaches the assay zone, the read beam of the drive assembly may be used to analyze various characteristics of the fluid (e.g., serum or clarified urine). The characteristics, which may be qualitative or quantitative in nature, may include at least one of the following: microsphere quantitation, colorimetric quantitation, microparticle agglutination, immuno precipitation, and reflectivity quantitation. The optical bio-disc may have encoded information including instructions for such analysis, or other analysis, of the fluid assay chamber. At the assay zone, the light may be transmitted or reflected to a detector to detect and/or measure some aspect of the fluid. In addition, the particulates could be at (or provided to) another assay zone (not shown) for investigation (e.g., to determine the hematocrit, which is the proportion, by volume, of the blood that consists of red blood cells).

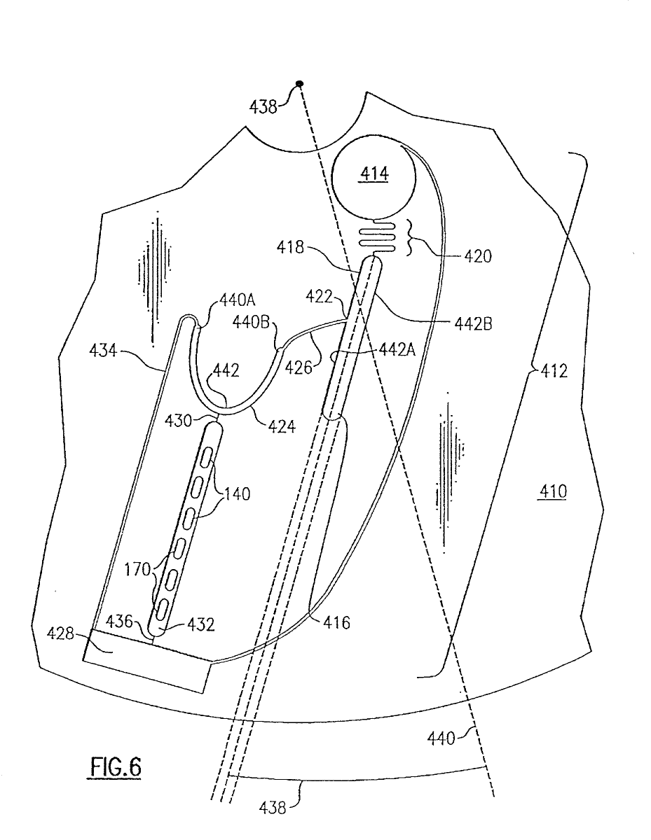

The apparatus described herein may be used in any of multiple applications. For example, FIG. 6 illustrates that assay zone 432 may include one or more target zones 140, 170 described above. The different target zones may be configured differently so that multiple different analyses may be performed on the same sample of serum supplied by the metering chamber.

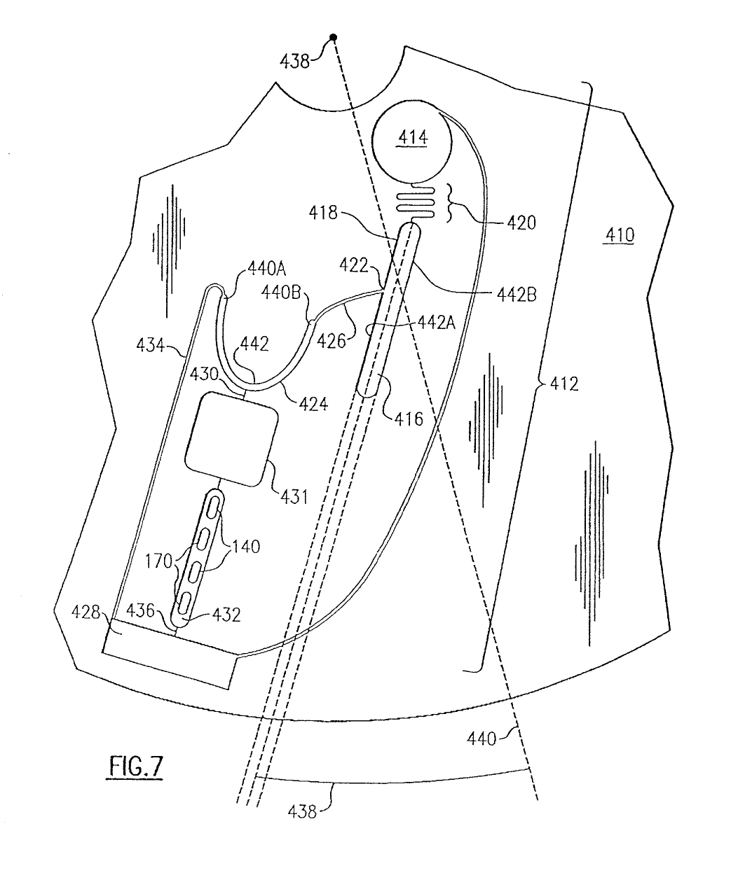

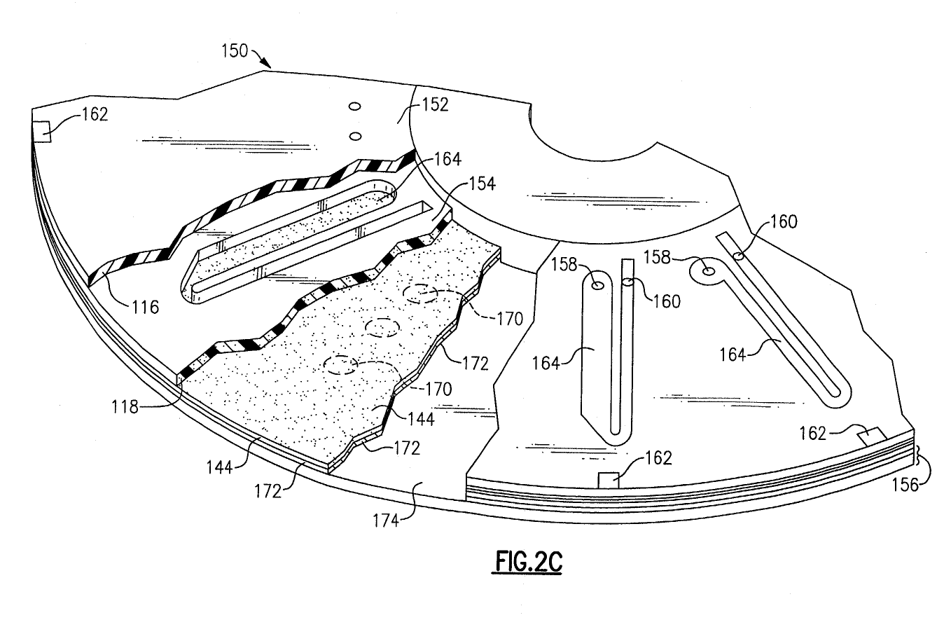

FIG. 7 illustrates that a reaction chamber 431 may be provided between the metering chamber and the assay zone to allow the metered fluid to react with an assay reagent, a bioactive agent, or another material before being directed further downstream to the assay zone. In some cases, all or part of the assay zone may serve as a reaction chamber in place of or in addition to reaction chamber 431. For example, depending on the performance of a particular bioactive agent in a particular procedure, the assay zone may or may not be suitable to serve as an effective reaction chamber.

For example, three different types (e.g., having different colors or sizes) of particles with three different types of bioactive agents (e.g., antibodies) attached thereto may be mixed with the fluid in the reaction chamber, and then the mixture may be directed to the assay zone. In such a case, if the assay zone has three different target areas bearing three different bioactive agents, the three different types of particles may collect or be captured in the three different target areas in preparation for detection and analysis. In a specific example, a fluid (e.g., blood serum) may have molecules of interest (e.g., particular protein molecules, prostate specific antigens), and a desired set of bioactive agent bearing particles in the reaction chamber may bind to the molecules of interest, forming molecule tagged particles. In such a case, when the molecule tagged particles encounter the target zones in the assay zone, the molecules of the molecule tagged particles also bind to a particular bioactive agent of a particular target zone, and thereby cause the molecule tagged particles to bind to and collect at the particular target zone, and become available for detection and analysis. The molecules of interest thus serve as bridges between different bioactive agents, forming bioactive agent sandwiches with the molecules of interest in the middle of each sandwich. Accordingly, the particle serves as a proxy for the molecule interest; detection of the particle is interpreted as detection of the molecule of interest.

In at least some cases, more than one bioactive agent sandwich is needed to retain a particle in a target zone against the rotation induced centrifugal force, which allows the detection of one particle to be interpreted as the detection of multiple molecules of interest. A calibration curve calculation may be used that derives the detected concentration of molecules of interest from the density of the bioactive agent on the particles and the detected concentration of the particles in the target zone.

Molecule detection reliability approaches certainty, since the desired set of particles collects at the particular target zone only if the molecules of interest are available to bind to both the bioactive agent of the desired set and the bioactive agent of the zone (e.g., if each of the bindings has an error rate of 1 in 10⁹, the error rate of using both bindings is 1 in 10¹⁸). A match to two bioactive agents is required for detection, which can be particularly important in certain cases. For example, one bioactive agent interface of the pregnancy hormone hCG is the same as one bioactive agent interface of the follicle-stimulating hormone (useful for fertility treatment), which necessitates, for distinguishing purposes, the use of additional bioactive agents that are sensitive to the other interfaces of the respective hormones.

If first and second target zones of the same type are used and the first zone is positioned upstream of the second zone, it may be expected that molecules of interest will be detected at a higher concentration in the first zone than in the second zone. In some cases, one target zone may be used in place of multiple different target zones, and conclusions may be drawn from analysis (e.g., color or size analysis) of the material that collects in the one target zone.

In at least some cases, material may be freeze-dried into an area of the circuit, such as the assay zone, one or more of the target zones, or the reaction chamber. The freeze-dried material may include an assay reagent or a bioactive agent and may dissolve upon interaction with a sample or a specimen. An advantage of using freeze-dried material is that the disc need not be removed from and re-inserted into the reader in an extra step solely for the purpose of introducing the assay reagent or bioactive agent. Another advantage of using freeze-dried material is that, in at least some cases, refrigeration and other preservation techniques and related equipment are unnecessary or less important, which renders at least some implementations of the invention more amenable to use in remote or resource-deprived locations or other places where preservation would otherwise be difficult or impossible.

For example, material that includes a bioactive agent bound to a particle may be freeze-dried into the assay zone or reaction chamber. In some or all cases, it may be advantageous to avoid using freeze-dried material in the metering chamber, because all or significant portions of such freeze-dried material may be washed out as fluid passes through the metering chamber to the waste chamber in advance of the point in the procedure in which fluid is directed downstream toward the assay zone from the metering chamber.

In any case involving freeze-dried materials, a procedure may allow sufficient time for the freeze-dried material to be dissolved and bound to molecules (or other small compositions of matter) of interest to an effective degree.

Some or all of the parts of one or more of the microfluidic circuits shown in FIGS. 4-7, including, for example, one or more of the antechamber 414, the separation chamber 418, the metering chamber 424, overflow conduit 434, assay zone 432, waste chamber 428, and conduits, junctions, and ends 420, 422, 426, 430, 432, 440A, 440B, may be formed by utilization of a channel layer such as channel layer 104 (FIGS. 1A-1C) when implemented in a reflective bio-disc or a channel layer such as channel layer 154 (FIGS. 2A-2B) when implemented in a transmissive bio-disc. In at least some embodiments, such parts or circuits in accordance with the present invention may be formed in the channel layer, which may include a plastic sheet being 25-100 microns in thickness.

In at least the case of an alternative implementation employing an optical bio-disc that lacks a channel layer 104 or 154 (e.g., a two-layer disc), some or all of the parts of one or more of the microfluidic circuits shown in FIGS. 4-7 may be formed in a cap layer or a substrate layer, wherein the cap layer is bonded directly to the substrate layer in the absence of a channel layer 104, 154 or other intervening layer. In a first embodiment of the alternative implementation, the microfluidic circuit is formed in the cap layer. In a second embodiment of the alternative implementation, the microfluidic circuit is formed in the substrate layer. In a third embodiment of the alternative implementation, portions of the microfluidic circuit are formed in the cap layer and other portions of the microfluidic circuit are formed in the substrate layer, and the portions achieve registration, suitably assembling the microfluidic circuit, when the cap and substrate layers are adhered together.

The term bioactive agent as used herein refers to any molecule A that recognizes a molecule B and binds with specificity thereto. The phrase “binds with specificity” is meant herein to refer to the binding of molecule A to molecule B to a significantly greater extent (e.g., by at least two fold, at least five fold, at least 10 fold, at least one hundred fold, or at least 1000 fold or more) relative to other molecules that may be present in a biological sample. For example, molecules that specifically recognize and bind to other molecules include antibodies, ligands, receptors, enzymes, substrates, biotin, and avidin. The bioactive agent used as described herein may be obtained from any source, including viral, bacterial, fungal, plant, animal, in vitro, or synthetically produced materials.

In at least some embodiments, the bioactive agent includes an antibody and the particle has at least one antibody bound thereto. As used herein, the term “antibody” includes polyclonal, monoclonal, and recombinantly created antibodies. Antibodies used as described herein can be produced in vivo or in vitro. Methods for the production of antibodies are well known to those skilled in the art. For example, see Antibody Production: Essential Techniques, Peter Delves (Ed.), John Wiley & Son Ltd, ISBN: 0471970107 (1997), which is incorporated herein in its entirely by reference. Alternatively, antibodies may be obtained from commercial sources, e.g., Research Diagnostics Inc., Pleasant Hill Road, Flanders, N.J. 07836.

The selection of a bioactive agent to be bound to a particle is within the skill of those in the art. For example, a receptor-specific ligand may be bound to a particle for the purpose of agglutinating cells expressing the receptor recognized by the ligand or a particle may be bound by a lectin that binds specifically a sugar moiety expressed on the surface of a select population of cells for the purpose of agglutinating those cells. Thus, the techniques and apparatus described herein are easily adapted to many biological assays.

The term “antibody” is not meant to be limited to antibodies of any one particular species; for example, antibodies of humans, mice, rats, and goats are all contemplated by the invention. The term “antibody” is also inclusive of any class or subclass of antibodies,. For example, the IgG antibody class may be used for agglutination purposes or, if a higher antibody polyvalency is desired, the IgD or IgM class of antibodies may be utilized for the same purpose. Antibody fragments can also be utilized as a bioactive agent of the invention. The use of antibodies in the art of medical diagnostics is well known to those skilled in the art. For example, see Diagnostic and Therapeutic Antibodies (Methods in Molecular Medicine), Andrew J. T. George and Catherine E. Urch (Eds.), Humana Press; ISBN: 0896037983 (2000) and Antibodies in Diagnosis and Therapy: Technologies, Mechanisms and Clinical Data (Studies in Chemistry Series), Siegfried Matzku and Rolf A. Stahel (Eds.), Harwood Academic Pub.; ISBN: 9057023105 (1999), which are incorporated herein in their entirely by reference.

The particle with the bioactive agent bound thereto can be structured in any suitable way. In at least some embodiments of the invention, one or more bioactive agents can be directly linked to the particle. Thus, particles may be uniformly bound with multiple copies of a single bioactive agent or, alternatively, particles may be bound with multiple copies of two or more bioactive agents to increase the specificity of a binding reaction or the occurrence of a subsequent reaction. In other embodiments, the bioactive agent can be indirectly linked to the particle. For example, a particle may be coated with a protein such as streptavidin and a bioactive agent such as an antibody can be linked to the streptavidin by way of a biotin moiety attached to the antibody.

With respect to at least some embodiments of the invention, the particle has a first bioactive agent bound thereto and the first bioactive agent binds a second bioactive agent. For example, an anti-IgM IgG antibody can serve as a first bioactive agent bound to a particle, which itself binds an IgM antibody, the second bioactive agent. Thus, the bioactive agent bound to a particle can in at least some embodiments include more than one bioactive agent linked to one another in tandem.

Other embodiments are within the scope of the following claims. For example, one or more of the circuit's substructures (e.g., the metering chamber) may have a different shape. The disc may use replaceable microfluidic circuits. The disc or microfluidic circuit may include a mechanism for delivering the sample to the antechamber automatically, e.g., upon rotation.

What is claimed is:

1. An optical bio-disc for use in separating components of particulate suspension, comprising:
a separation chamber for containing a particulate suspension having a fluid component and a particulate matter component;
a fluid metering chamber communicating with the separation chamber by a first conduit, the first conduit having an entry point at the separation chamber, the entry point being accessible to the fluid component when the bio-disc is rotated causing separation of the fluid component and the particulate matter component in the separation chamber; and
a fluid assay chamber communicating with the fluid metering chamber by a second conduit.

2. The optical bio-disc of claim 1, wherein the fluid metering chamber has an elongated shape and the axis of rotation is radially closer to opposite ends of the fluid metering chamber than the axis of rotation is to the middle of the fluid metering chamber.

3. The optical bio-disc of claim 1, wherein opposite boundaries of the separation chamber are more than 10 degrees and less than 90 degrees in the same direction from a vector of rotation-induced centrifugal force extending through the center of the separation chamber.

4. The optical bio-disc of claim 1, further comprising:
an overflow chamber communicating with the fluid metering chamber by a third conduit.

5. The optical bio-disc of claim 1, further comprising:
an overflow chamber communicating with the fluid assay chamber by a third conduit.

6. The optical bio-disc of claim 1, wherein the fluid metering chamber has a U shape.

7. The optical bio-disc of claim 1, wherein the optical bio-disc has encoded information including instructions for analysis of the fluid assay chamber.

8. The optical bio-disc of claim 1, wherein the optical bio-disc has encoded information including instructions for controlling rotation of the bio-disc.

9. The optical bio-disc of claim 1, further comprising:
an antechamber in communication with the separation chamber by a third conduit.

10. The optical bio-disc of claim 9, wherein the third conduit includes first and second legs carrying particulate suspension in substantially opposite directions.

11. An optical bio-disc, comprising:
a substrate having encoded information associated therewith, the encoded information being readable by a disc drive assembly to control rotation of the disc;
an antechamber associated with the substrate;
a separation tube associated with the substrate, the separation tube in fluid communication with the antechamber;
a metering chamber associated with the substrate, the metering chamber in fluid communication with the separation tube; and
an assay zone associated with the substrate, the assay zone in fluid communication with the metering chamber so that when a particulate suspension including a particulate matter component and a liquid component is deposited into the antechamber, rotating the substrate in a predetermined manner delivers a metered amount of the liquid component to the assay zone.

12. An optical bio-disc, comprising:
a substrate having a center and an outer edge;
an antechamber associated with the substrate;
a separation tube associated with the substrate, the separation tube in fluid communication with the antechamber;
a metering chamber associated with the substrate, the metering chamber in fluid communication with the separation tube;
an assay zone associated with the substrate, the assay zone in fluid communication with the metering chamber; and
a waste chamber associated with the substrate, the waste chamber in fluid communication with the meeting chamber so that when a particulate suspension including a particulate matter component and a liquid component is deposited into the antechamber, rotating the substrate in a predetermined manner delivers a metered amount of the liquid component to the assay zone while an excess amount of the liquid component is delivered to the waste chamber.

13. The optical bio-disc of claim 12 wherein the substrate includes encoded information readable by a disc drive assembly to control rotation of the disc.

14. The optical bio-disc of claim 11 wherein the disc drive assembly includes a read beam enabled to analyze the liquid component in the assay zone.

15. A fluidic circuit in a substrate for separating and metering a liquid component of a particulate suspension from particulate matter associated therewith, the fluidic circuit comprising:
an antechamber;
a separation tube in fluid communication with the antechamber;
a metering chamber in fluid communication with the separation tube;
an assay zone in fluid communication with the metering chamber; and
a waste chamber in fluid communication with the meeting chamber so that when a particulate suspension including a particulate matter component and a liquid component is deposited into the antechamber, causing the particulate suspension flow through the separation tube and the metering chamber delivers a metered amount of the liquid component to the assay zone while an excess amount of the liquid component is delivered to the waste chamber.

16. The fluidic circuit of claim 15 wherein the particulate suspension includes a blood sample, the particulate matter component includes at least one from the group of white blood cells and red blood cells, and the liquid component includes serum.

17. The fluidic circuit of claim 15 wherein the particulate suspension includes a urine sample, the particulate matter component includes at least one from the group of epithelial cells, casts, and bacteria, and the liquid component includes clarified urine.

18. The fluidic circuit of claim 15 wherein the particulate suspension includes an environmental water sample, the particulate matter component includes at least one from the group of dirt, biological matter, particulate contaminants, and bacteria, and the liquid component includes clarified water.

19. The fluidic circuit of claim 16 wherein when the disc is processed in an optical drive, a read beam is directed at the assay zone to analyze the serum.

20. The fluidic circuit of claim 17 wherein when the disc is processed in an optical drive, a read beam is directed at the assay zone to analyze the clarified urine.

21. The fluidic circuit of claim 18 wherein when the disc is processed in an optical drive, a read beam is directed at the assay zone to analyze the clarified water.

22. The fluidic circuit of claim 15 wherein the particulate suspension includes a blood sample, the particulate matter component includes at least one from the group of white blood cells and red blood cells, and the liquid component includes serum.

23. The fluidic circuit of claim 15 wherein the particulate suspension includes a urine sample, the particulate matter component includes at least one from the group of epithelial cells, casts, and bacteria, and the liquid component includes clarified urine.

24. The fluidic circuit of claim 15 wherein the particulate suspension includes an environmental water sample, the particulate matter component includes at least one from the group of dirt, biological matter, and particulate contaminants, and the liquid component includes clarified water.

25. The fluidic circuit of claim 22 wherein when the disc is processed in an optical drive, a read beam is directed at the assay zone to analyze the serum.

26. The fluidic circuit of claim 23 wherein when the disc is processed in an optical drive, a read beam is directed at the assay zone to analyze the clarified urine.

27. The fluidic circuit of claim 24 wherein when the disc is processed in an optical drive, a read beam is directed at the assay zone to analyze the clarified water.

28. An optical bio-disc drive assembly, comprising:
a motor for rotating a respective optical bio-disc;
means for controlling the motor so that the bio-disc is rotated in a predetermined manner;
a source of electromagnetic radiation;
means for focusing the electromagnetic radiation at a predetermined location on the optical bio-disc;
means for receiving a return beam returned from an assay zone associated with the bio-optical disc, the assay zone including a liquid component of a particulate suspension which was separated from particulate matter associated therewith by rotating the optical bio-disc in the predetermined manner; and
means for analyzing the return beam so that desired characteristics of the liquid component are determined.

29. The drive assembly of claim 28 further including means for displaying results from analyzing the return beam.

30. The optical bio-disc of claim 11 wherein the particulate suspension includes a sample of amniotic fluid, the particulate matter component includes at least one from the group of sloughed cells, cell debris, cells, vernix, and bacteria, and the liquid component includes clarified amniotic fluid.

31. The optical bio-disc of claim 11 wherein the particulate suspension includes a sample of cerebrospinal fluid, the particulate matter component includes at least one from the group of cell debris, cells, clots, and bacteria, and the liquid component includes clarified cerebrospinal fluid.

32. The optical bio-disc of claim 11 wherein the particulate suspension includes a sample of synovial fluid, the particulate matter component includes at least one from the group of cell debris, cells, clots, and bacteria, and the liquid component includes clarified synovial fluid.

33. The optical bio-disc of claim 11 wherein the particulate suspension includes a sample of pleural fluid, the particulate matter component includes at least one from the group of cell debris, cells, lipid, and bacteria, and the liquid component includes clarified pleural fluid.

34. The optical bio-disc of claim 11 wherein the particulate suspension includes a sample of pericardial fluid, the particulate matter component includes at least one from the group of cell debris, cells, lipid, and bacteria, and the liquid component includes clarified pericardial fluid.

35. The optical bio-disc of claim 11 wherein the particulate suspension includes a sample of peritoneal fluid, the particulate matter component includes at least one from the group of cell debris, cells, lipid, and bacteria, and the liquid component includes clarified peritoneal fluid.

36. A rotatable disc for use in separating components of particulate suspension, comprising:
an separation chamber, elongated along a line, for containing a particulate suspension having a fluid component and a particulate matter component, the separation chamber being oriented at an angle that is in a range of 0-45 degrees relative to a vector of rotation-induced centrifugal force extending through the center of the separation chamber; and
a fluid metering chamber communicating with the separation chamber by an entry point at the separation chamber, the entry point being accessible to the fluid component when the bio-disc is rotated causing separation of the fluid component and the particulate matter component in the separation chamber.

37. A rotatable disc for use in separating components of particulate suspension, comprising:
a metering chamber formed in a substrate of the disc for receiving a liquid during a first rotation phase, and for delivering the liquid to another chamber in the substrate in a second rotating phase.

38. The disc of claim 37, wherein the metering chamber is U-shaped with a bight portion at the outermost radial point of the metering chamber.

39. The disc of claim 37, wherein the metering chamber is substantially symmetric about a radius from an axis of rotation of the disc.

40. A method for use in separating components of particulate suspension, comprising:
at a first sustained speed, rotating an apparatus including a fluidic circuit having a separation component for holding the particulate suspension, a metering component communicating with the separation component, and an assay component communicating with the metering component, wherein centrifugal force resulting from the first sustained speed is insufficient to cause fluid of the particulate suspension to move from the separation component to the metering component;
rotating the apparatus at a second sustained speed causing centrifugal force that is sufficient to cause the fluid to move from the separation component to the metering component and being insufficient to cause the fluid to move from the metering component to the assay component; and
rotating the apparatus at a third sustained speed causing centrifugal force that is sufficient to cause the fluid to move from the metering component to the assay component.

41. An optical disc for separating components of particulate suspension, comprising:
a main chamber having a separation chamber for containing a particulate suspension having a fluid component and a particulate matter component and a fluid metering chamber communicating with the separation chamber by a first conduit, the first conduit having an entry point at the separation chamber, the entry point being accessible to the fluid component when the bio-disc is rotated causing separation of the fluid component and the particulate matter component in the separation chamber;
a reaction chamber in communication with the main chamber; and
a capture area in communication with the reaction chamber.

42. An optical disc for separating components of particulate suspension, comprising:
a main chamber;
a reaction chamber in communication with the main chamber, the reaction chamber having a separation chamber for containing a particulate suspension having a fluid component and a particulate matter component and a fluid metering chamber communicating with the separation chamber by a first conduit, the first conduit having an entry point at the separation chamber, the entry point being accessible to the fluid component when the bio-disc is rotated causing separation of the fluid component and the particulate matter component in the separation chamber; and
a capture area in communication with the reaction chamber.

43. A rotatable disc for use in separating components of particulate suspension, comprising:
an separation chamber, elongated along a line, for containing a particulate suspension having a fluid component and a particulate matter component, the separation chamber being oriented at an angle of approximately 30 degrees relative to a vector of rotation-induced centrifugal force extending through the center of the separation chamber; and
a fluid metering chamber communicating with the separation chamber by an entry point at the separation chamber, the entry point being accessible to the fluid component when the bio-disc is rotated causing separation of the fluid component and the particulate matter component in the separation chamber.

44. The optical bio-disc of claim 1, wherein the antechamber includes freeze-dried material.

45. The optical bio-disc of claim 1, wherein the antechamber includes anticoagulant.

46. The optical bio-disc of claim 1, wherein the freeze-dried material includes anticoagulant.

47. The optical bio-disc of claim 1, wherein the fluid metering chamber is configured to retain a controlled amount of fluid after termination of fluid flow through the fluid metering chamber.

48. The optical bio-disc of claim 1, wherein the location of the entry point at the separation chamber corresponds to an expected composition of the particulate suspension.

49. The optical bio-disc of claim 1, wherein the location of the entry point at the separation chamber corresponds to an expected volume of particulate matter component in the separation chamber.

50. The optical bio-disc of claim 1, wherein the fluid assay chamber further comprises a target zone bearing a bioactive agent.

51. The optical bio-disc of claim 1, further comprising a reaction chamber communicating with the fluid metering chamber.

52. The optical bio-disc of claim 1, further comprising a reaction chamber communicating with the fluid assay chamber.

53. The optical bio-disc of claim 1, wherein the fluid assay chamber further comprises first and second target zones bearing respective sets of bioactive agents, the first target zone being disposed upstream of the second target zone.

54. The optical bio-disc of claim 1, wherein the fluid assay chamber includes freeze-dried material.

55. The optical bio-disc of claim 51, wherein the reaction chamber includes freeze-dried material.

56. The optical bio-disc of claim 1, wherein the fluid assay chamber includes a bioactive agent.

57. The optical bio-disc of claim 51, wherein the reaction chamber includes a bioactive agent.
